(19) United States
(12) Patent Application Publication
(10) Pub. No.: US 2021/0038061 A1
(43) Pub. Date: Feb. 11, 2021

(54) INSUFFLATION DEVICE AND METHOD OF CONTROLLING INSUFFLATION DEVICE

(71) Applicant: OLYMPUS CORPORATION, Tokyo (JP)

(72) Inventors: Takefumi UESUGI, Tokyo (JP); Koji YAMAOKA, Tokyo (JP); Yuma KASUYA, Tokyo (JP); Shinya TORII, Tokyo (JP); Keita KIMURA, Tokyo (JP); Kunitoshi HIRAGA, Tokyo (JP)

(73) Assignee: OLYMPUS CORPORATION, Tokyo (JP)

(21) Appl. No.: 17/077,536

(22) Filed: Oct. 22, 2020

Related U.S. Application Data
(63) Continuation of application No. PCT/JP2019/003127, filed on Jan. 30, 2019.

(30) Foreign Application Priority Data
Apr. 24, 2018 (JP) 2018-083283

(51) Int. Cl. A61B 1/015; A61B 1/00; A61B 1/313

(52) U.S. Cl. CPC A61B 1/015; A61B 1/3132; A61B 1/00068

(57) ABSTRACT

An insufflation device includes: a gas feeding conduit through which a gas is supplied to a body cavity of a patient; a first open/close valve mounted on the gas feeding conduit, the first open/close valve being an electromagnetic valve configured to control a flow rate of the gas flowing through the gas feeding conduit by pulse width modulation; and a controller, the controller being configured to: detect a current value of a current which flows through the first open/close valve; calculate a resistance value of the first open/close valve based on the current value; determine a duty ratio of a pulse signal applied to the first open/close valve based on the resistance value and a target gas feeding flow rate of the gas supplied to a body cavity; and supply the pulse signal, an effective voltage of which is adjusted, to the first open/close valve.

### CROSS REFERENCE TO RELATED APPLICATION

This application is a continuation application of PCT/JP2019/003127 filed on Jan. 30, 2019 and claims benefit of Japanese Application No. 2018-083283 filed in Japan on Apr. 24, 2018, the entire contents of which are incorporated herein by this reference.

### BACKGROUND OF THE INVENTION

### 1. Field of the Invention

Embodiments of the present invention relate to an insufflation device which supplies a gas into an abdominal cavity, a lumen or the like in endoscopic surgery, and a method of controlling an insufflation device.

### 2. Description of the Related Art

Recently, for the purpose of alleviating invasiveness to a patient, laparoscopic surgery which performs therapeutic treatment without opening an abdominal cavity is performed. In the laparoscopic surgery, a first trocar which guides, for example, an endoscope for observation into a body cavity, and a second trocar which guides a treatment instrument to a part to be treated are inserted into an abdominal portion of a patient. In the laparoscopic surgery, using the endoscope inserted into the abdominal cavity through an insertion hole of the first trocar, treatment or the like is performed while observing the part to be treated and the treatment instrument inserted through an insertion hole of the second trocar.

In such laparoscopic surgery, for the purpose of ensuring a field of view of the endoscope and for the purpose of ensuring a region for operating the treatment instrument, an insufflation device is used. The insufflation device increases a pressure in the body cavity to a predetermined pressure by injecting an insufflation gas such as a carbon dioxide gas into the body cavity thus ensuring the field of view of the endoscope and the region for operating the treatment instrument (for example, see Japanese Patent Application Laid-Open Publication No. 2016-52478).

Conventionally, for adjusting a gas feeding flow rate of an insufflation device, a proportional control valve which can continuously change a degree of opening corresponding to a current value is used in general. In the proportional control valve, a current value changes corresponding to a change in temperature of a coil incorporated in the proportional control valve. To prevent a change in gas feeding flow rate caused by a change in a current value, usually, a constant current circuit is used in a control of degree of opening of the proportional control valve.

### SUMMARY OF THE INVENTION

According to an aspect of the present invention, there is provided an insufflation device which includes: a gas feeding conduit through which a gas is supplied to a body cavity of a patient; a first open/close valve mounted on the gas feeding conduit, the first open/close valve being an electromagnetic valve configured to control a flow rate of the gas flowing through the gas feeding conduit by pulse width modulation; and a controller. The controller is configured to: detect a current value of a current which flows through the first open/close valve; calculate a resistance value of the first open/close valve based on the current value; determine a duty ratio of a pulse signal applied to the first open/close valve based on the resistance value and a target gas feeding flow rate of the gas supplied to the body cavity; and supply the pulse signal, an effective voltage of which is adjusted, to the first open/close valve.

According to another aspect of the present invention, there is provided a method of controlling an insufflation device which includes: detecting a current value of a current which flows through a first open/close valve which is an electromagnetic valve mounted on a gas feeding conduit through which a gas is supplied to a body cavity of a patient; calculating a resistance value of the first open/close valve based on the current value; determining a duty ratio of a pulse signal applied to the first open/close valve based on the resistance value and a target gas feeding flow rate of the gas supplied to the body cavity; and applying the pulse signal, an effective voltage of which is adjusted, to the first open/close valve.

### BRIEF DESCRIPTION OF THE DRAWINGS

FIG. 1 is a view for describing one example of an overall configuration of an insufflation device according to an embodiment of the present invention;

FIG. 2 is a flowchart for describing one example of control steps of a proportional control valve according to a first embodiment;

FIG. 3 is a view for describing one example of a gas feeding flow rate characteristic of the proportional control valve according to the first embodiment;

FIG. 4A is a view for describing one example of a control table corresponding to the gas feeding flow rate characteristic;

FIG. 4B is a view for describing one example of a control table corresponding to the gas feeding flow rate characteristic;

FIG. 4C is a view for describing one example of a control table corresponding to the gas feeding flow rate characteristic;

FIG. 4D is a view for describing one example of a control table corresponding to the gas feeding flow rate characteristic;

FIG. 5 is a view for describing one example of an overall configuration of an insufflation device according to a second embodiment;

FIG. 6 is a flowchart for describing one example of control steps of a proportional control valve according to the second embodiment;

FIG. 7 is a view for describing one example of a gas feeding flow rate characteristic of the proportional control valve according to the second embodiment;

FIG. 8A is a view for describing one example of a control table corresponding to the gas feeding flow rate characteristic;

FIG. 8B is a view for describing one example of a control table corresponding to the gas feeding flow rate characteristic;

FIG. 8C is a view for describing one example of a control table corresponding to the gas feeding flow rate characteristic, and

FIG. 8D is a view for describing one example of a control table corresponding to the gas feeding flow rate characteristic.

### DETAILED DESCRIPTION OF THE PREFERRED EMBODIMENTS

Embodiments of the present invention are described with reference to drawings hereinafter.

### First Embodiment

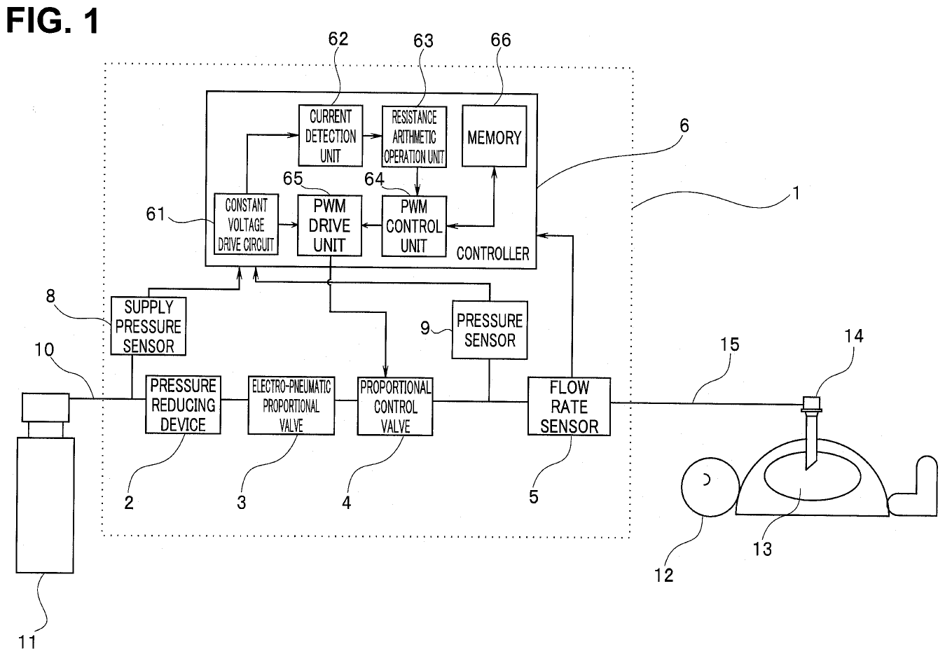

FIG. 1 is a view for describing one example of an overall configuration of an insufflation device 1 according to an embodiment of the present invention. As shown in FIG. 1, in the insufflation device 1 according to the embodiment, a pressure reducing device 2, an electro-pneumatic proportional valve 3, a proportional control valve 4, a flow rate sensor 5, a supply pressure sensor 8, a pressure sensor 9, and a control unit (controller) 6 are mainly disposed.

A gas supply source 11 (for example, a carbon dioxide gas cylinder) is connected to the insufflation device 1 through a high pressure gas tube 10. To the insufflation device 1, a gas feeding tube 15 which forms a gas feeding conduit for feeding an insufflation gas such as a carbon dioxide gas into a body cavity through a trocar 14 inserted into an abdominal cavity 13 of a patient 12 is connected. On the gas feeding tube 15, the pressure reducing device 2, the electro-pneumatic proportional valve 3, the proportional control valve 4, the flow rate sensor 5, the supply pressure sensor 8 and the pressure sensor 9 are mounted.

The pressure reducing device 2 reduces a pressure of a high pressure gas supplied from the gas supply source 11 to a pressure at a level which does not endanger a human body. For example, a pressure of a gas supplied at a high pressure of approximately 6 MPa from the gas supply source 11 is reduced to a pressure of approximately 6 to 600 kPa.

The electro-pneumatic proportional valve 3 which forms a second open/close valve is a kind of an electromagnetic drive valve, and is configured to adjust a gas feeding pressure to a predetermined pressure value by electrically adjusting a degree of opening of a valve element in multiple stages. A pressure of a carbon dioxide gas, a pressure of which is reduced by the pressure reducing device 2, is changed to a gas feeding pressure within a range of approximately 0 to 80 mmHg based on a control signal inputted from the control unit 6. However, in the embodiment, the electro-pneumatic proportional valve 3 changes the gas feeding pressure to a predetermined pressure which is set in advance.

The proportional control valve 4 which forms a first open/close valve is a kind of an electromagnetic drive valve, and is an adjusting valve which uses an electromagnetic coil as a drive unit. A magnetic force is generated when a current is supplied to the electromagnetic coil so that a plunger is attracted, and opening/closing of the valve is performed. The proportional control valve 4 is configured such that a degree of opening of a valve element is controlled by controlling the position of the plunger based on a magnitude of a current which flows through the electromagnetic coil so that a flow rate of a gas which flows through the gas feeding conduit can be adjusted to a predetermined value.

The flow rate sensor 5 measures a flow rate of a carbon dioxide gas supplied into a body cavity, and outputs a measurement result to the control unit 6.

The supply pressure sensor 8 measures a pressure in the gas feeding conduit. The measurement of the pressure is performed at the time of supplying a gas. A pressure of the gas supplied from the gas supply source 11 is measured, and a measurement result is outputted to the control unit 6.

The pressure sensor 9 measures a pressure in the abdominal cavity 13 through the gas feeding tube 15. The measurement of the pressure is performed during a time period where the feeding of a gas is stopped. A measurement result of the pressure sensor 9 is outputted to the control unit 6.

The gas feeding tube 15 is a tube which guides a gas fed from the insufflation device 1 to the trocar 14. In general, the gas feeding tube 15 is formed using a material having flexibility, and has a length of approximately 3 m.

The control unit 6 performs a control of the respective constitutional parts in the insufflation device 1. In FIG. 1, among components in the control unit 6, only the constitutional parts relating to the adjustment of a gas feeding flow rate are indicated. Hereinafter, the constitutional parts shown in FIG. 1 are described. The control unit 6 includes a constant voltage drive circuit 61, a current detection unit 62, a resistance arithmetic operation unit 63, a PWM control unit 64, a PWM drive unit 65, and a memory 66.

The plurality of components in the control unit 6 (the resistance arithmetic operation unit 63, the PWM (pulse width modulation) control unit 64 and the PWM drive unit 65) may be each constituted of a processor operated by software or may be each constituted of hardware. For example, the constant voltage drive circuit 61 and the current detection unit 62 are respectively formed of hardware. The resistance arithmetic operation unit 63, the PWM control unit 64 and the PWM drive unit 65 are each constituted of a processor operated by software. The memory 66 is a semiconductor memory.

The constant voltage drive circuit 61 supplies a direct current signal of a constant voltage to the PWM drive unit 65.

The current detection unit 62 detects a current which flows through the electromagnetic coil of the proportional control valve 4.

The resistance arithmetic operation unit 63 which forms a resistance value calculation unit calculates a resistance value of the electromagnetic coil which constitutes the proportional control valve 4. More specifically, the resistance arithmetic operation unit 63 calculates a resistance value R by dividing a voltage value which the constant voltage drive circuit 61 supplies by a current value detected by the current detection unit 62.

The PWM control unit 64 estimates a gas feeding flow rate characteristic of the proportional control valve 4 (a gas feeding flow rate characteristic with respect to a PWM duty ratio) based on the resistance value R calculated by the resistance arithmetic operation unit 63. A current which flows through the proportional control valve 4 changes depending on a temperature of the electromagnetic coil incorporated in the proportional control valve 4. When the temperature of the electromagnetic coil is increased, the resistance value of the electromagnetic coil is increased and hence, in a case where a signal applied to the proportional control valve 4 is a direct current signal of a constant voltage, a current value of a current which flows through the electromagnetic coil is lowered. When the current value is lowered, a magnetic force generated in the electromagnetic coil is decreased and hence, a degree of opening of the proportional control valve 4 becomes small. In other words, in a case where a signal applied to the electromagnetic coil is a direct current signal of a constant applied voltage, along with the increase of a resistance value of the electromagnetic coil, a flow rate of a gas which flows through the gas feeding conduit is lowered. In view of the above, the adjustment is performed such that, when a pulse rectangular wave signal of a constant peak voltage is applied to the electromagnetic coil, a PWM duty ratio is controlled corresponding to a resistance value of the electromagnetic coil so that an effective voltage of a signal applied to the proportional control valve is changed whereby a predetermined gas feeding flow rate is obtained. For example, when a duty ratio (a ratio of a pulse width with respect to one cycle of a pulse signal) is 50%, the effective voltage becomes 50%.

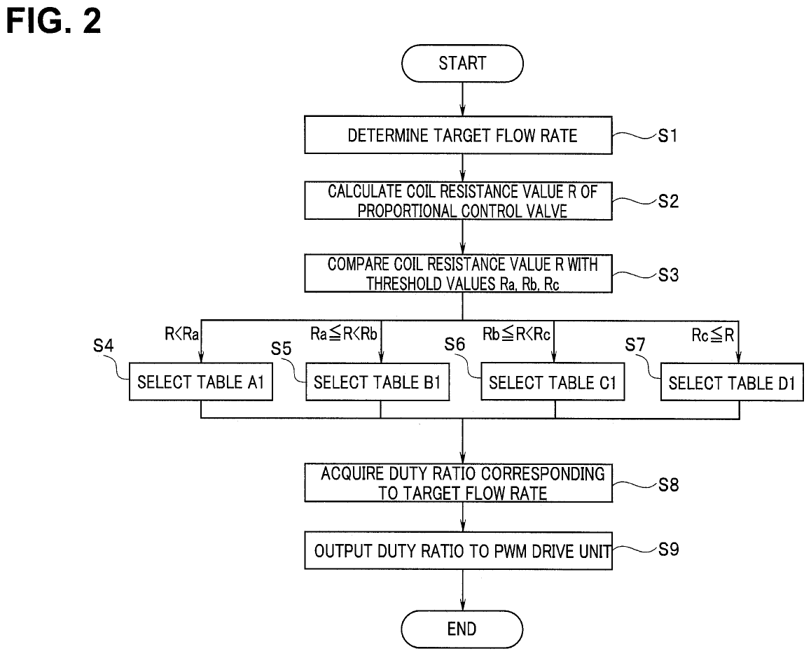

A control method of PWM (duty ratio) is described hereinafter. FIG. 2 is a flowchart for describing one example of control steps of the proportional control valve according to the first embodiment. First, the control unit 6 determines a target gas feeding flow rate (S1). Next, the resistance arithmetic operation unit 63 calculates a resistance value R of the electromagnetic coil which constitutes the proportional control valve 4 (S2).

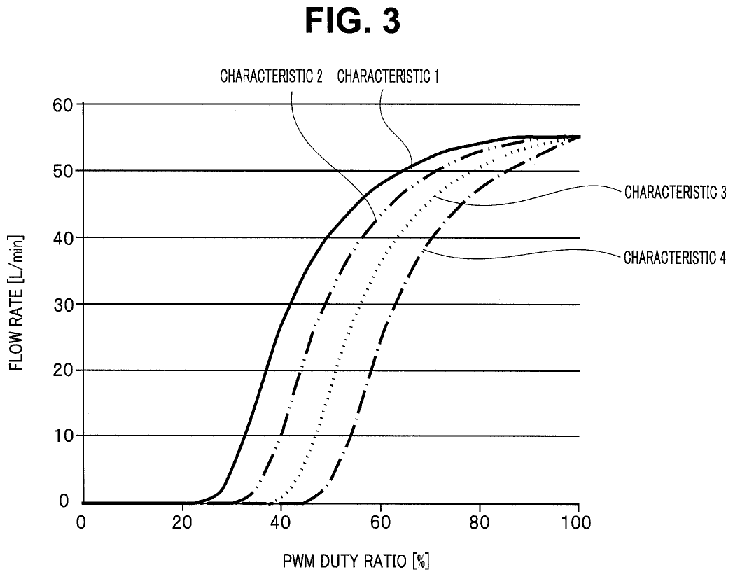

Subsequently, the PWM control unit 64 estimates a gas feeding flow rate characteristic of the proportional control valve based on the resistance value R of the electromagnetic coil. FIG. 3 is a view for describing one example of a gas feeding flow rate characteristic of the proportional control valve according to the first embodiment. In FIG. 3, a PWM duty ratio of a signal applied to the proportional control valve 4 is taken on a horizontal axis, and a flow rate of a gas which flows through the gas feeding conduit is taken on a vertical axis. Along with the increase of a resistance value of the electromagnetic coil, the gas feeding flow rate characteristic translates in a positive direction on the horizontal axis while having an approximately same function shape. Accordingly, as shown in FIG. 3, for example, four kinds of gas feeding flow rate characteristics of a characteristic 1 to a characteristic 4 corresponding to ranges of the resistance values are set in advance.

In other words, as threshold values of resistance values of the electromagnetic coil which become selection references of the gas feeding flow rate characteristic, three kinds of threshold values Ra, Rb, Rc (Ra<Rb<Rc) are set. A graph indicating the characteristic 1 is set as a gas feeding flow rate characteristic when the resistance value R is set to R<Ra, and a graph indicating the characteristic 2 is set as the gas feeding flow rate characteristic when the resistance value R is set to Ra≤R<Rb. Further, a graph indicating the characteristic 3 is set as the gas feeding flow rate characteristic when the resistance value R is set to Rb≤R<Rc, and a graph indicating a characteristic 4 is set as the gas feeding flow rate characteristic when the resistance value R is set to Rc≤R.

The PWM control unit 64 compares the resistance value R calculated by the resistance arithmetic operation unit 63 with three kinds of resistance threshold values (Ra, Rb, Rc) (S3). When the resistance value R is set to R<Ra, the PWM control unit 64 estimates that the gas feeding flow rate characteristic of the proportional control valve 4 is the graph indicating the characteristic 1. When the resistance value R is set to Ra≤R<Rb, the PWM control unit 64 estimates that the gas feeding flow rate characteristic of the proportional control valve 4 is the graph indicating the characteristic 2. When the resistance value R is set to Rb≤R<Rc, the PWM control unit 64 estimates that the gas feeding flow rate characteristic of the proportional control valve 4 is the graph indicating the characteristic 3. Further, when the resistance value R is set to Rc≤R, the PWM control unit 64 estimates that the gas feeding flow rate characteristic of the proportional control valve 4 is the graph indicating the characteristic 4.

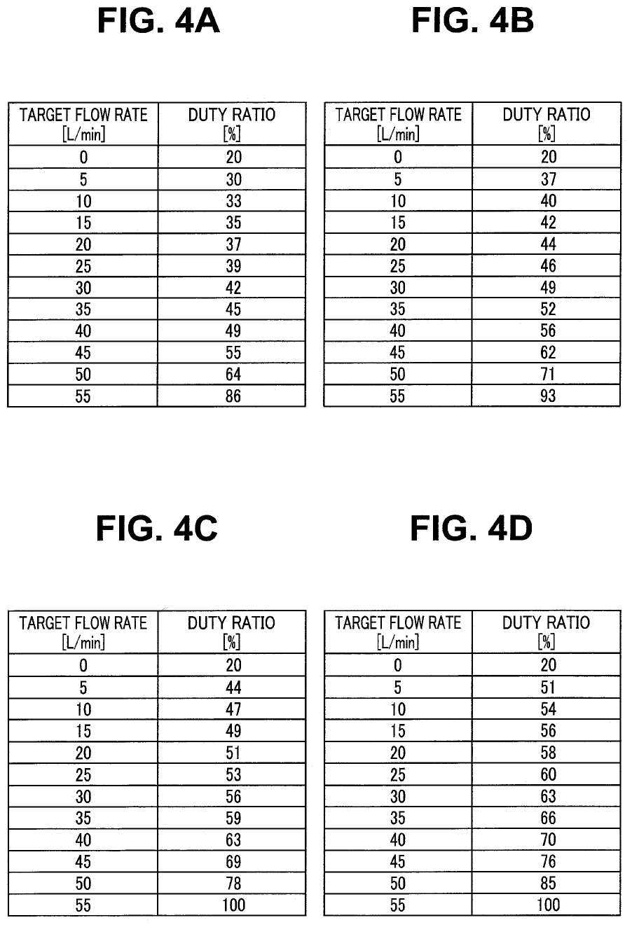

In the memory 66, for every gas feeding flow rate characteristic, a PWM duty ratio with respect to the target flow rate is registered. In other words, in the memory 66, the gas feeding flow rate characteristics which respectively correspond to four different resistance value ranges are set. FIG. 4A, FIG. 4B, FIG. 4C and FIG. 4D are views for describing one example of control tables corresponding to the gas feeding flow rate characteristics. FIG. 4A is the table which corresponds to the characteristic 1 and shows a PWM duty ratio with respect to a target gas feeding flow rate (the table being referred to as table A1 hereinafter). FIG. 4B is the table which corresponds to the characteristic 2 and shows a PWM duty ratio with respect to a target gas feeding flow rate (the table being referred to as table B1 hereinafter). FIG. 4C is the table which corresponds to the characteristic 3 and shows a PWM duty ratio with respect to a target gas feeding flow rate (the table being referred to as table C1 hereinafter). FIG. 4D is the table which corresponds to the characteristic 4 and shows a PWM duty ratio with respect to a target gas feeding flow rate (the table being referred to as table D1 hereinafter).

Subsequently, the PWM control unit 64 looks up the control table corresponding to the estimated gas feeding flow rate characteristic. For example, when the PWM control unit 64 estimates that the gas feeding flow rate characteristic is the characteristic 1, the PWM control unit 64 looks up the table A1 shown in FIG. 4A (S4). When the PWM control unit 64 estimates that the gas feeding flow rate characteristic is the characteristic 2, the PWM control unit 64 looks up the table B1 shown in FIG. 4B (S5). When the PWM control unit 64 estimates that the gas feeding flow rate characteristic is the characteristic 3, the PWM control unit 64 looks up the table C1 shown in FIG. 4C (S6). When the PWM control unit 64 estimates that the gas feeding flow rate characteristic is the characteristic 4, the PWM control unit 64 looks up the table D1 shown in FIG. 4D (S7).

Then, in the looked-up table, the PWM control unit 64 acquires a PWM duty ratio which corresponds to the target gas feeding flow rate set in S1 (S8). For example, when the looked-up table is the table A1 and the target gas feeding flow rate is 10 L/min, 33% is extracted as the PWM duty ratio corresponding to the target gas feeding flow rate. When the looked-up table is the table B1 and the target gas feeding flow rate is 10 L/min, 40% is extracted as the PWM duty ratio corresponding to the target gas feeding flow rate.

Lastly, the PWM control unit 64 outputs the extracted PWM duty ratio to the PWM drive unit 65 (S9).

The PWM drive unit 65 performs an ON/OFF pulse control with respect to a direct current signal of a voltage inputted from the constant voltage drive circuit 61 in accordance with a PWM duty ratio inputted from the PWM control unit 64, and applies a pulse signal to the proportional control valve 4.

In this manner, according to the embodiment, the PWM control unit 64 selects a gas feeding flow rate characteristic corresponding to a resistance value R of the electromagnetic coil which constitutes the proportional control valve 4, and sets a PWM duty ratio for realizing a target gas feeding flow rate based on the gas feeding flow rate characteristic. Accordingly, even in a case where a signal is applied to the proportional control valve 4 using a constant voltage circuit, an effective voltage can be adjusted to a pulse signal having a proper duty ratio corresponding to a resistance value R, and hence, even when a resistance value R of the electromagnetic coil changes due to a change in temperature, a gas feeding flow rate can be controlled with high accuracy.

In closing the proportional control valve 4, the PWM control unit 64 does not set a PWM duty ratio to zero, and sets a PWM duty ratio which falls within an insensitive band of the proportional control valve 4. More specifically, in the table A1 for the characteristic 1, a PWM duty ratio (=20%) when a target gas feeding flow rate is 0 L/min is set. By setting a PWM duty ratio in this manner, a voltage is applied to the proportional control valve 4 even the proportional control valve 4 is a closed state and hence, a state where a current flows through the electromagnetic coil can be constantly maintained whereby a resistance value R can be calculated.

In the above-mentioned case, as a threshold value of a resistance value of the electromagnetic coil which is used as a selection reference of a gas feeding flow rate characteristic, three kinds of threshold values Ra, Rb, Rc are set, and four kinds of gas feeding flow rate characteristics are set. However, the number of kinds of the threshold values and the number of kinds of gas feeding flow rate characteristics are not limited to the above-mentioned numbers. For example, a case is considered where one kind of threshold value is set and only two kinds of gas feeding flow rate characteristics are set. A case is also considered where three or more kinds of threshold values are set and four or more kinds of gas feeding flow rate characteristics are set. It is preferable that at least four kinds of different gas feeding flow rate characteristics be set in the memory 66.

### Second Embodiment

In the insufflation device 1 according to the first embodiment described above, a gas feeding flow rate is controlled by adjusting a degree of opening of the proportional control valve 4. On the other hand, the embodiment differs from the first embodiment with respect to a point that a gas feeding flow rate is more finely controlled by also adjusting a degree of opening of an electro-pneumatic proportional valve 3 in addition to a control of a degree of opening of a proportional control valve 4.

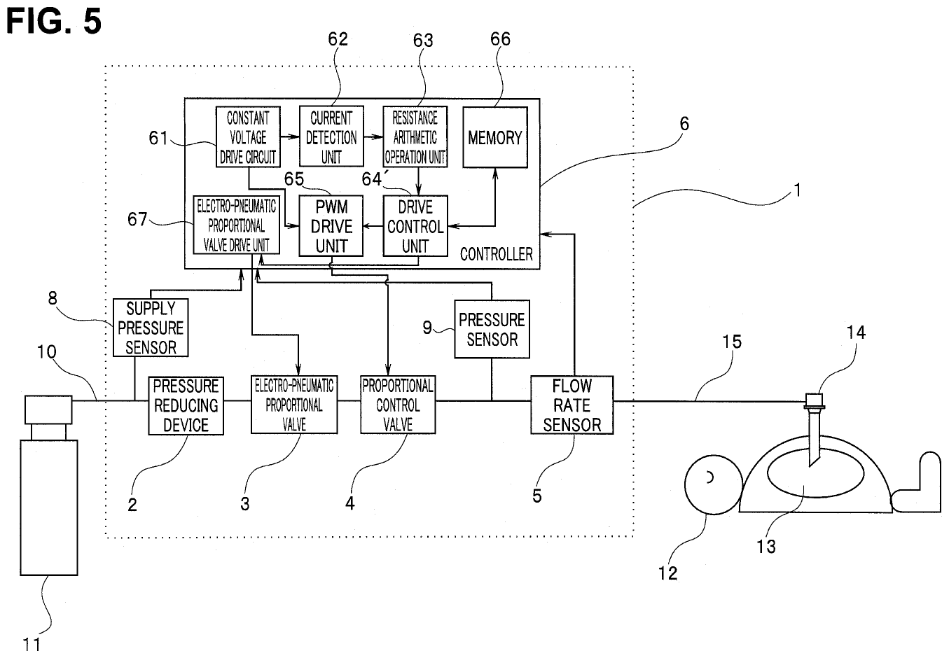

Hereinafter, the configuration of an insufflation device according to the embodiment is described with reference to FIG. 5. FIG. 5 is a view for describing one example of an overall configuration of the insufflation device according to a second embodiment. With respect to the configuration of the embodiment, components of the insufflation device are substantially equal to the components of insufflation device according to the first embodiment except the control unit 6. Substantially identical components are given the same symbols and the detailed description of the components is omitted.

The control unit 6 which forms a control unit further includes an electro-pneumatic proportional valve drive unit 67 in addition to a constant voltage drive circuit 61, a current detection unit 62, a resistance arithmetic operation unit 63, a PWM control unit 64′, a PWM drive unit 65, and a memory 66.

At least one of the PWM control unit 64′ and the electro-pneumatic proportional valve drive unit 67 may be constituted of a processor which is operated by software or may be constituted of hardware.

The PWM control unit 64′ controls a PWM duty ratio corresponding to a resistance value R calculated by the resistance arithmetic operation unit 63, and changes an effective voltage of a signal applied to a proportional control valve 4. The PWM control unit 64′ also changes an output of an electro-pneumatic proportional valve 3 corresponding to a target gas feeding flow rate. In other words, the PWM control unit 64′ performs the adjustment such that a predetermined gas feeding flow rate is obtained by controlling a degree of opening of the proportional control valve 4 and a degree of opening of the electro-pneumatic proportional valve 3.

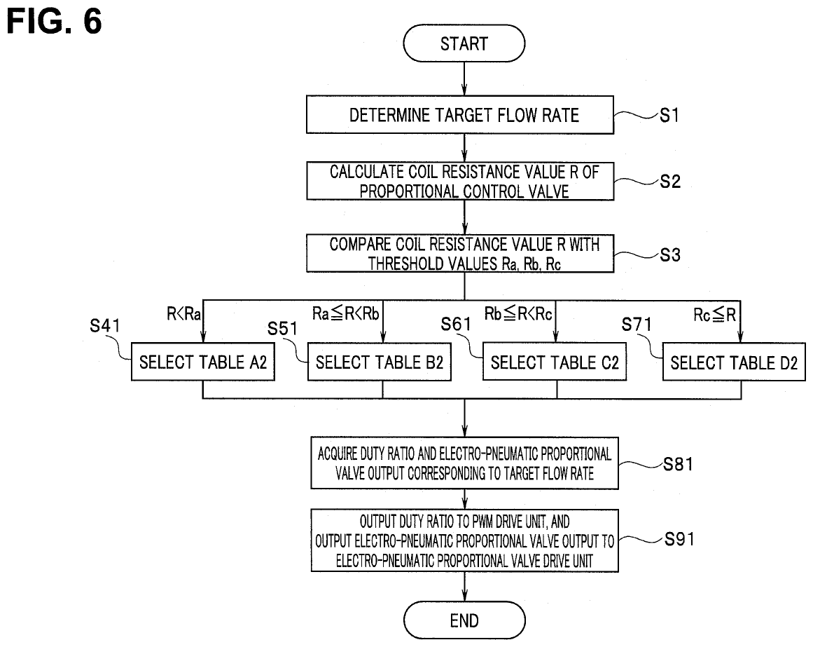

A method of controlling a PWM duty ratio in the embodiment is described hereinafter. FIG. 6 is a flowchart for describing one example of control steps of the proportional control valve according to the second embodiment. First, the control unit 6 determines a target gas feeding flow rate (S1). Next, the resistance arithmetic operation unit 63 calculates a resistance value R of the electromagnetic coil which constitutes the proportional control valve 4 (S2).

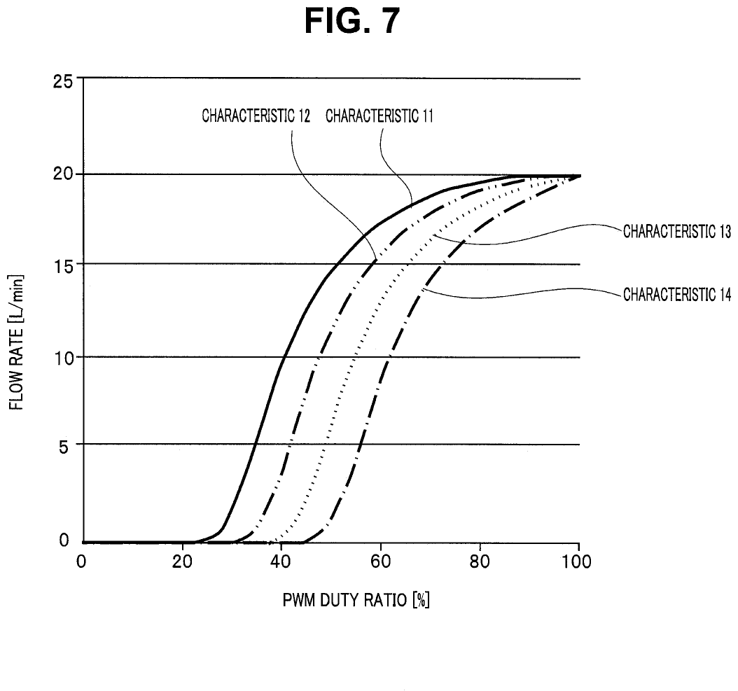

Subsequently, the PWM control unit 64′ estimates a gas feeding flow rate characteristic of the proportional control valve based on the resistance value R of the electromagnetic coil. FIG. 7 is a view for describing one example of a gas feeding flow rate characteristic of the proportional control valve according to the second embodiment. In FIG. 7, a PWM duty ratio of a voltage applied to the proportional control valve 4 is taken on a horizontal axis, and a flow rate of a gas which flows through the gas feeding conduit is taken on a vertical axis. Along with the increase of a resistance value of the electromagnetic coil, the gas feeding flow rate characteristic translates in a positive direction on the horizontal axis while having an approximately same function shape. Accordingly, as shown in FIG. 7, for example, four kinds of gas feeding flow rate characteristics of a characteristic 11 to a characteristic 14 corresponding to ranges of the resistance values are set in advance.

In other words, as threshold values of resistance values of the electromagnetic coil which become selection references of the gas feeding flow rate characteristic, three kinds of threshold values Ra, Rb, Rc (Ra<Rb<Rc) are set. A graph indicating the characteristic 11 is set as a gas feeding flow rate characteristic when the resistance value R is set to R<Ra, and a graph indicating the characteristic 12 is set as the gas feeding flow rate characteristic when the resistance value R is set to Ra≤R<Rb. Further, a graph indicating the characteristic 13 is set as the gas feeding flow rate characteristic when the resistance value R is set to Rb≤R<Rc, and a graph indicating the characteristic 14 is set as the gas feeding flow rate characteristic when the resistance value R is set to Rc≤R.

The PWM control unit 64′ compares the resistance value R calculated by the resistance arithmetic operation unit 63 with three kinds of resistance threshold values (Ra, Rb, Rc) (S3). When the resistance value R is set to R<Ra, the PWM control unit 64′ estimates that the gas feeding flow rate characteristic of the proportional control valve 4 is the graph indicating the characteristic 11. When the resistance value R is set to Ra≤R<Rb, the PWM control unit 64′ estimates that the gas feeding flow rate characteristic of the proportional control valve 4 is the graph indicating the characteristic 12. When the resistance value R is set to Rb≤R<Rc, the PWM control unit 64′ estimates that the gas feeding flow rate characteristic of the proportional control valve 4 is the graph indicating the characteristic 13. Further, when the resistance value R is set to Rc≤R, the PWM control unit 64′ estimates that the gas feeding flow rate characteristic of the proportional control valve 4 is the graph indicating the characteristic 14.

The gas feeding flow rate characteristic shown in FIG. 7 is compared with the gas feeding flow rate characteristic shown in FIG. 3. When the PWM duty ratio is set to 100%, a gas feeding flow rate is 50 L/min in the gas feeding flow rate characteristic shown in FIG. 3, while a gas feeding flow rate is 20 L/min in the gas feeding flow rate characteristic shown in FIG. 7. In other words, in the embodiment, when a target gas feeding flow rate falls within a range of 0 to 20 L/min, a gas feeding flow rate can be controlled by adjusting a degree of opening of the proportional control valve 4 by controlling a PWM duty ratio. When the target gas feeding flow rate exceeds 20 L/min, a control is made such that the gas feeding flow rate becomes a target flow rate by fully opening the proportional control valve 4 by setting the PWM duty ratio to 100% and by adjusting a degree of opening of the electro-pneumatic proportional valve 3 by controlling an output value of the electro-pneumatic proportional valve.

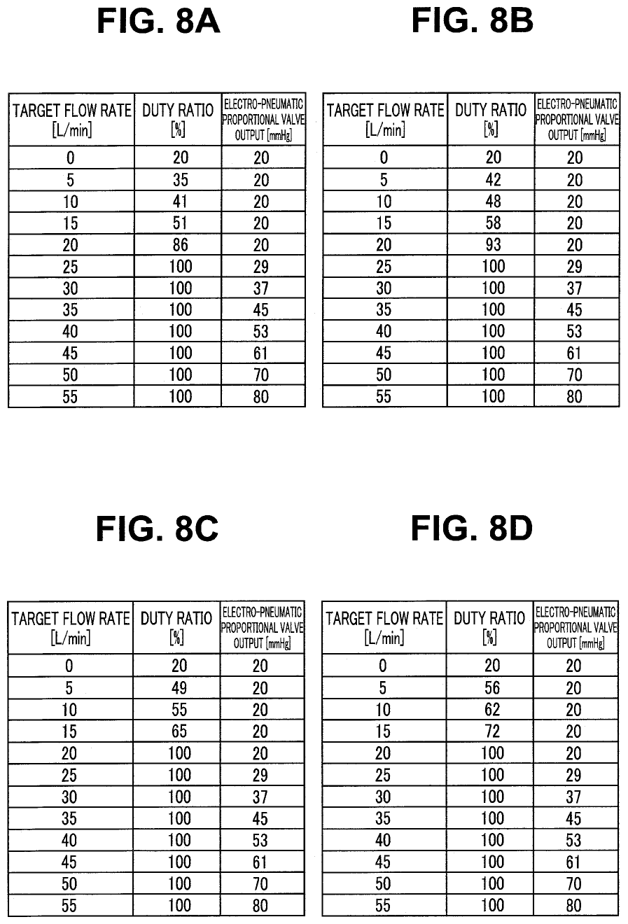

In the memory 66, for every gas feeding flow rate characteristic, a PWM duty ratio and an output value of the electro-pneumatic proportional valve with respect to the target flow rate are registered. FIG. 8A, FIG. 8B, FIG. 8C and FIG. 8D are views for describing one example of control tables corresponding to the gas feeding flow rate characteristics. FIG. 8A is the table which corresponds to the characteristic 11 and shows a PWM duty ratio and an output value of the electro-pneumatic proportional valve with respect to a target gas feeding flow rate (the table being referred to as table A2 hereinafter). FIG. 8B is the table which corresponds to the characteristic 12 and shows a PWM duty ratio and an output value of the electro-pneumatic proportional valve with respect to a target gas feeding flow rate (the table being referred to as table B2 hereinafter). FIG. 8C is the table which corresponds to the characteristic 13 and shows a PWM duty ratio and an output value of the electro-pneumatic proportional valve with respect to a target gas feeding flow rate (the table being referred to as table C2 hereinafter). FIG. 8D is the table which corresponds to the characteristic 14 and shows a PWM duty ratio and an output value of the electro-pneumatic proportional valve with respect to a target gas feeding flow rate (the table being referred to as table D2 hereinafter).

Subsequently, the PWM control unit 64′ looks up the control table corresponding to the estimated gas feeding flow rate characteristic. For example, when the PWM control unit 64′ estimates that the gas feeding flow rate characteristic is the characteristic 11, the PWM control unit 64′ looks up the table A2 shown in FIG. 8A (S41). When the PWM control unit 64′ estimates that the gas feeding flow rate characteristic is the characteristic 12, the PWM control unit 64′ looks up the table B2 shown in FIG. 8B (S51). When the PWM control unit 64′ estimates that the gas feeding flow rate characteristic is the characteristic 13, the PWM control unit 64′ looks up the table C2 shown in FIG. 8C (S61). When the PWM control unit 64′ estimates that the gas feeding flow rate characteristic is the characteristic 14, the PWM control unit 64′ looks up the table D2 shown in FIG. 8D (S71).

Then, in the looked-up table, the PWM control unit 64′ acquires a PWM duty ratio and an output value of the electro-pneumatic proportional valve which correspond to the target gas feeding flow rate set in S1 (S81). For example, when the looked-up table is the table A2 and the target gas feeding flow rate is 40 L/min, 100% is extracted as the PWM duty ratio corresponding to the target gas feeding flow rate, and 53 mmHg is extracted as an output of the electro-pneumatic proportional valve. When the looked-up table is the table B2 and the target gas feeding flow rate is 10 L/min, 48% is extracted as the PWM duty ratio corresponding to the target gas feeding flow rate, and 20 mmHg is extracted as an output of the electro-pneumatic proportional valve.

Lastly, the PWM control unit 64′ outputs the extracted PWM duty ratio to the PWM drive unit 65, and outputs the extracted output value of the electro-pneumatic proportional valve to the electro-pneumatic proportional valve drive unit 67 (S91).

The electro-pneumatic proportional valve drive unit 67 which forms a valve drive unit generates a drive signal in accordance with an output value of the electro-pneumatic proportional valve inputted from the PWM control unit 64′, and controls a degree of opening of the electro-pneumatic proportional valve 3.

In this manner, according to the embodiment, the PWM control unit 64′ selects a gas feeding flow rate characteristic corresponding to a resistance value R of the electromagnetic coil which constitutes the proportional control valve 4, and sets a PWM duty ratio and an output value of the electro-pneumatic proportional valve 3 for realizing a target gas feeding flow rate based on the gas feeding flow rate characteristic. In other words, when a target gas feeding flow rate is small (for example, when a capacity of a cavity which becomes an object to which insufflation is applied is small, such as insufflation via a rectum), a PWM duty ratio is adjusted in a state where an output value of the electro-pneumatic proportional valve 3 is set to a small value. On the other hand, when a target gas feeding flow rate is large, an output value of the electro-pneumatic proportional valve 3 is adjusted in a state where a PWM duty ratio is fixed to 100%. Accordingly, it is possible to control a gas feeding flow rate with more accuracy in a range where a gas feeding flow rate to which the flow rate adjustment is applied using a PWM duty ratio is small.

Although several embodiments of the present invention have been described heretofore, the embodiments are exemplified as examples, and the embodiments do not intend to limit the scope of the present invention. The novel embodiments can be carried out in various other modes, and various omissions, replacements and alterations can be made without departing from the gist of the present invention. These embodiments and the modifications are included in the scope and the gist of the present invention, and are included in the invention described in claims and the scope equivalent to the invention.

What is claimed is:

1. An insufflation device comprising:
a gas feeding conduit through which a gas is supplied to a body cavity of a patient;
a first open/close valve mounted on the gas feeding conduit, the first open/close valve being an electromagnetic valve configured to control a flow rate of the gas flowing through the gas feeding conduit by pulse width modulation; and
a controller, the controller being configured to:
detect a current value of a current which flows through the first open/close valve;
calculate a resistance value of the first open/close valve based on the current value;
determine a duty ratio of a pulse signal applied to the first open/close valve based on the resistance value and a target gas feeding flow rate of the gas supplied to the body cavity; and
supply the pulse signal, an effective voltage of which is adjusted, to the first open/close valve.

2. The insufflation device according to claim 1, wherein the controller further includes a memory in which a plurality of gas feeding flow rate characteristics indicating a correspondence relationship between the target gas feeding flow rate and the duty ratio are set, and
the controller is configured to select an optimum gas feeding flow rate characteristic from the plurality of gas feeding flow rate characteristics based on the resistance value, and is configured to identify the duty ratio corresponding to the target gas feeding flow rate with respect to the selected gas feeding flow rate.

3. The insufflation device according to claim 2, wherein at least four different gas feeding flow rate characteristics are set in the memory.

4. The insufflation device according to claim 1, further comprising a second open/close valve mounted on the gas feeding conduit, the second open/close valve being configured to control a flow rate of the gas flowing through the gas feeding conduit,
wherein the controller is configured to adjust a degree of opening of the second open/close valve based on the resistance value and the target gas feeding flow rate of the gas supplied to the body cavity and to drive the second open/close valve.

5. The insufflation device according to claim 4, wherein the controller further includes a memory in which a plurality of gas feeding flow rate characteristics indicating a correspondence relationship among the target gas feeding flow rate, the duty ratio and a set value for adjusting the degree of opening of the second open/close valve to a predetermined degree of opening are set, and
the controller is configured to select an optimum gas feeding flow rate characteristic from the plurality of gas feeding flow rate characteristics based on the resistance value, to identify the duty ratio corresponding to the target gas feeding flow rate with respect to the selected gas feeding flow rate characteristic, and to drive the second open/close valve using the set value corresponding to the target gas feeding flow rate with respect to the selected gas feeding flow rate characteristic.

6. The insufflation device according to claim 5, wherein in each of the gas feeding flow rate characteristics, when the target gas feeding flow rate is equal to or less than a first gas feeding flow rate, the set value is set to a predetermined value and the duty ratio is set to a value which corresponds to the target gas feeding flow rate, and when the target gas feeding flow rate exceeds the first gas feeding flow rate, the duty ratio is set to a predetermined value and the set value is set to a value which corresponds to the target gas feeding flow rate.

7. The insufflation device according to claim 5, wherein at least four different gas feeding flow rate characteristics are set in the memory.

8. The insufflation device according to claim 6, wherein at least four different gas feeding flow rate characteristics are set in the memory.

9. The insufflation device according to claim 4, wherein the second open/close valve is an electro-pneumatic proportional valve.

10. The insufflation device according to claim 5, wherein the second open/close valve is an electro-pneumatic proportional valve.

11. The insufflation device according to claim 6, wherein the second open/close valve is an electro-pneumatic proportional valve.

12. The insufflation device according to claim 7, wherein the second open/close valve is an electro-pneumatic proportional valve.

13. The insufflation device according to claim 8, wherein the second open/close valve is an electro-pneumatic proportional valve.

14. A method of controlling an insufflation device comprising:
detecting a current value of a current which flows through a first open/close valve which is an electromagnetic valve mounted on a gas feeding conduit through which a gas is supplied to a body cavity of a patient;
calculating a resistance value of the first open/close valve based on the current value;
determining a duty ratio of a pulse signal applied to the first open/close valve based on the resistance value and a target gas feeding flow rate of the gas supplied to the body cavity; and
applying the pulse signal, an effective voltage of which is adjusted, to the first open/close valve.
